(19) United States
(12) Patent Application Publication
(10) Pub. No.: US 2025/0333437 A1
(43) Pub. Date: Oct. 30, 2025

(54) USE OF NON-INFORMATIONALAMINO ACID CHAINS TO MODIFY THE SOLUBILITY PROPERTIES OF PEPTIDES

(71) Applicants: Robert COIFMAN, MILLVILLE, NJ (US); Catherine YANG, Elk Grove, CA (US)

(72) Inventors: Robert COIFMAN, MILLVILLE, NJ (US); Catherine YANG, Elk Grove, CA (US)

(21) Appl. No.: 18/849,510

(22) PCT Filed: Mar. 6, 2023
(86) PCT No.: PCT/US2023/063784

§ 371 Date: Sep. 22, 2024

Related U.S. Application Data
(60) Provisional application No. 63/322,986, filed on Mar. 23, 2022.

(51) Int. Cl. C07K 1/107; A61K 39/00; A61K 39/015; A61K 39/04; A61K 39/215; C07K 14/00

(52) U.S. Cl. CPC C07K 1/1075; A61K 39/0008; A61K 39/0011; A61K 39/015; A61K 39/04; A61K 39/215; C07K 14/001

(57) ABSTRACT

Provided are methods of modifying the solubility properties of individual peptides and sets of peptides of which an example embodiment includes sets of overlapping peptides prepared to be suitable for use as vaccines to modulate the immune responses of recipients. The peptides are modified to have the requisite solubility properties for tissue delivery by precipitation without extraneous binding and modification of antigenicity. According to example embodiments. the modified peptides are suitable to be used in TDBP/VDBP methods. Also provided herein are the solubility-modified peptides themselves and kits that include them. Further provided are methods of administering solubility-modified peptides with the requisite solubility properties for tissue delivery by precipitation.

### FIELD

The present disclosure relates to methods of modifying the solubility properties of individual peptides and sets of peptides of which an example embodiment includes sets of overlapping peptides prepared to be suitable for use as vaccines to modulate the immune responses of recipients. Solubility modification and method of solubility modification are important for applications in which either or both of ability to administer a diagnostic or therapeutic peptide or set of peptides and ability to achieve an intended outcome depend on solubility and/or the way the structure of each affected native peptide is altered by the process of solubility modification.

### BACKGROUND

The therapeutic value of depositing a substance of interest within a volume of a target tissue of interest by precipitation from a water-miscible solvent in which it was administered is diluted by available water in the recipient tissue, was demonstrated for the urushiol of poison ivy in the field of allergy (Coifman RE, Yang CF, Tolerance to poison ivy following vaccine delivery by precipitation, Annals of Allergy, Asthma and Immunology 2019(Mar);122:331-33). More than 100 years of efforts by dozens of investigators failed to induce tolerance in individuals who were already sensitized to poison ivy or highly cross-reactive poison oak (Watson S: Toxicodendron hyposensitization programs, Clinics in Dermatology 1986;4(2):160-170., Kim Y, Flamm A, EISohly M, Kaplan DH et al: Poison Ivy, Oak, and Sumac Dermatitis: What Is Known and What Is New? Dermatitis. May/June 2019;30(3):183-190. doi: 10.1097/DER.0000000000000472), until the present inventors precipitated vaccines in micron-sized particles after intramuscular injection in small volumes of ethanol.

Immunomodulation in the opposite direction, from tolerance to sensitization for tumor antigens in cancer and from naiveté to protective sensitization in infectious disease, involves the same immune response switching mechanism except that the switches are thrown in the opposite direction (O'mahony L, Akdis M, Crameri R &Akdis C A: Novel immunotherapeutic approaches for allergy and asthma. Autoimmunity, November 2010; 43(7): 493-503 q Informa UK, Ltd. ISSN 0891-6934 print/1607-842X online DOI: 10.3109/08916931003674725). Delivery of antigen in the form of particles in the 0.5 to 5 micron size outperformed delivery of the same antigen in a water-soluble form for both immunomodulation from sensitization to tolerance (Neimert-Andersson T, Thunberg S, Swedin L, Wiedermann U, Jacobsson-Elunan G. Dahlen S.-E. Scheynius A, Gronlund H, van Hage M and, Gafvelin G: Carbohydrate-based particles reduce allergic inflammation in a mouse model for cat allergy. Allergy 2008:63:518-526) and from naiveté to sensitization (Kovacsovics-Bankowski M, Clark K, Benacerraf B, Rock K L. Efficient major histocompatibility complex class I presentation of exogenous antigen upon phagocytosis by macrophages. Proc Nail Acad Sci USA 1993;90:4942-4946).

### BRIEF SUMMARY OF THE INVENTION

The present inventors discovered the phenomenon of tissue deposition by precipitation (TDBP) with an allergy vaccine that was a catechol and naturally had the requisite solubility properties to achieve TDBP. In particular, the poison ivy antigen that induced tolerance when precipitated in muscle by precipitation is a catechol which is naturally insoluble in water and for which effective doses are soluble in small volumes of the pharmaceutically acceptable water-miscible solvent ethanol. The inventors termed the phenomenon vaccine delivery by precipitation (VDBP) which is a preferred embodiment of TDBP and they interpreted its probable mechanism of action.

Many antigens to which immunomodulation in either direction would be therapeutic, are proteins that do not have those solubility properties. The inventors further discovered ways to expand the range of applications of this method of feeding antigen to the immune system by imparting the requisite solubility properties on overlapping peptide derives of clinically relevant protein antigens. The immunomodulatory activity of protein antigens can be replicated with sets of their overlapping peptides, which in their native form are incapable of tissue deposition by precipitation (TDBP) because they also lack the requisite solubilities.

The present invention is not based on the biology of the immune system but on the physical chemistry of solubility. Potential embodiments include, but are not limited to, the delivery of antigen to modify the response of the immune system. They include the synthesis of modified versions of any individual peptides or sets of peptides for which those modifications will give them the solubility properties needed for tissue deposition by precipitation and for applications in which those modifications will not impede their intended physical or biological activity.

The scope of applications of this invention is also not limited to solubility modifications needed to enable TDBP. A non-limiting embodiment of solubility modification to improve water solubility would be for overlapping peptide vaccines intended for administration as aqueous solutions but for which the informationally significant AA sequence of one or more of the native overlapping peptides is insoluble in water.

### BRIEF DESCRIPTION OF THE FIGURE

FIG. 1 is a graphic representation of the function of the adaptive immune system in the skin, reproduced from Teunissen MBM, editor, Intradermal Immunization, Current Topics in Microbiology and Immunology Volume 351, pp. 113-138, Springer, Heidelberg, ISSN 0070-217X, ISBN 978-3-642-23689-1 e-ISBN978-3-642-23690-7 DOI 10.1007/978-3-642-23690-7. Dendritic cells (DCs) control the development of distinct T-cell responses. After internalization of environmental antigens, DCs migrate to the skin-draining lymph node while undergoing a process of maturation to acquire the unique capacity to prime naive T cells (Tn). The different DC subsets in homeostatic tissues and additional DC subsets in inflammatory conditions are indicated on the left site. The antigenic stimulus, the lineage of dendritic cells presenting the antigen, the cytokine milieu and possibly also the pre-stimulus state of the system are integrated in this figure into four signals (antigen presentation, costimulation, polarization, and homing directions-indicated in the blue boxes) that direct the maturation of naïve T cells (Tn) into the different classes of mature T cells shown in the figure (and probably others classes not yet known).

### DETAILED DESCRIPTION OF THE INVENTION

The present inventors discovered the process of Tissue Deposition by Precipitation (TDBP) with an allergy vaccine, for which they called the process Vaccine Delivery by Precipitation (VDBP). Therefore, VDBP is a set of embodiments of TDBP.

### Definitions

Informationally significant peptide: String of amino acids linked by peptide bonds for which the specific sequence of peptides in the string determines intended physiologic &/or immunologic activity.

Informationally insignificant peptide: String of amino acids for which the intended physiologic and/or immunologic activity is NOT dependent on the specific sequence of peptides in the string but on some other property such as effect on solubility.

Informationally significant amino acid (AA) sequence: Informationally significant portion(s) of a peptide that contains both informationally significant and informationally insignificant strings of amino acids.

Informationally insignificant AA sequence: Informationally insignificant portion(s) of a peptide that contains both informationally significant and informationally insignificant strings of amino acids.

A vaccine is defined by the CDC as “A product that stimulates a person's immune system to produce immunity to a specific disease, protecting the person from that disease.” Vaccines act by either reinforcing an existing state of immune system responsiveness or triggering immunomodulation from one state of responsiveness to another. Vaccines to protect patients from allergic diseases are designed to induce immunomodulation from pathological states of sensitization to immunological tolerance. Vaccines to protect against infectious diseases can either induce immunomodulation from immunological naiveté to protective sensitization or boost or enhance an existing state of protective sensitization. Vaccines to protect against cancer are designed to induce immunomodulation from tolerance of a patient's own cancer cells to a state of protective immunity.

While most vaccines are currently given by mouth or by subcutaneous or intramuscular injection, the skin is becoming a target of interest for the administration of vaccines to protect against both infectious and neoplastic diseases. This is done to exploit the presence and organization within the skin of cells and cell types whose ability to facilitate protective sensitization reflects the evolutionary role of the skin of protecting the tissues that live inside it from infection. The nasal mucosa has a similar evolutionary role and as such may also be an effective target tissue for immunomodulation from tolerance or naiveté to protective sensitization.

The particle size distribution in each individual application or embodiment of TDBP will be determined by the rate of solvent dilution. Particulate vaccines in the size range between 0.5 and 5 microns outperformed soluble versions of the same active ingredients for both immunomodulation from sensitization to tolerance (Neimert-Andersson T, Thunberg S, Swedin L, Wiedermann U, Jacobsson-Elunan G, Dahlen S.-E. Scheynius A, Gronlund H, van Hage M and, Gafvelin G: Carbohydrate-based particles reduce allergic inflammation in a mouse model for cat allergy. Allergy 2008:63:518-526) and from naiveté to protective sensitization (Kovacsovics-Bankowski M, Clark K, Benacerraf B, Rock KL. Efficient major histocompatibility complex class I presentation of exogenous antigen upon phagocytosis by macrophages. Proc Nat Acad Sci USA 1993;90:4942-4946). Particulate vaccines in this size range are efficiently taken up by naïve dendritic antigen presenting cells by a process called micropinocytosis (Xiang SD, Scholzen A, Minigo G, et al. Pathogen recognition and development of particulate vaccines: does size matter? Methods. 2006;40:1e9). The present inventors serendipitously hit this sweet spot in their initial attempt to make a poison ivy allergy vaccine for a single sensitive and occupationally exposed patient. With the refinement of their formulations, and dosing schedules for the precipitation of hundreds of thousands to millions of micron-sized particles of its water-insoluble antigen within a volume of a recipient tissue as a water-miscible solvent in which the antigen was administered was diluted by the water content of the recipient tissue, 90% of treated patients experienced durable and measurable clinically relevant tolerance to previously not tolerated levels of exposure. Of the small number who lost or failed to achieve initial tolerance and requested retreatment 100% responded to either a single booster or a second course of treatment (See Coifman RE, Yang CF, Tolerance to poison ivy following vaccine delivery by precipitation, Annals of Allergy, Asthma and Immunology 2019(Mar);122:331-33).

The unprecedented immunomodulatory potency achieved when an antigen (with 100 years of failure when delivered by other routes) was precipitated into hundreds of thousands to millions of micron-sized particles within a volume of a properly chosen recipient tissue, led the inventors to recognize and discover the mechanism resulting in the beneficial results, and discover that beneficial outcomes might be achieved by the same method of delivery of other therapeutic substances. The class of other therapeutic substances for which the present invention enables the same method of delivery are peptides, with a preferred, but non-limiting, embodiment being peptides synthesized to contain the antigenic epitopes of clinically relevant antigenic proteins but with integrated C-terminal and N-terminal strings of additional hydrophobic amino acids (AAs) to impart the necessary solubility properties.

The immunologically active segments of proteins (epitopes) trigger immunomodulation by presentation to naive T lymphocytes bound to major histocompatibility complex (MHC) class II peptides on the surfaces of antigen-presenting dendritic cells (APC's). Conformational epitopes that trigger immunological reactions are comprised of multiple short linear amino acid chains (Berglund L, Andrade J, Odeberg J and Uhle M: The epitope space of the human proteome. Protein Science (2008), 17:606-613.). Therapeutic immunomodulation to conformational epitopes should therefore be achievable by dendritic cell MHC class II presentation of their component linear sequences.

Overlapping peptides of protein antigens of known amino acid sequence can be made by solid phase synthesis based on modeling of selected 3D epitopes, for which there are open source methods (Stawikowski M & Fields GB: Introduction to Peptide Synthesis. Curr Protoc Protein Sci. 2002 February; CHAPTER: Unit-18.1. doi: 10.1002/0471140864.ps1801s26, Coin, I., Beyermann, M. & Bienert, M. Solid-phase peptide synthesis: from standard procedures to the synthesis of difficult sequences. Nat Protoc 2, 3247-3256 (2007). https://doi.org/10.1038/nprot 2007.454).

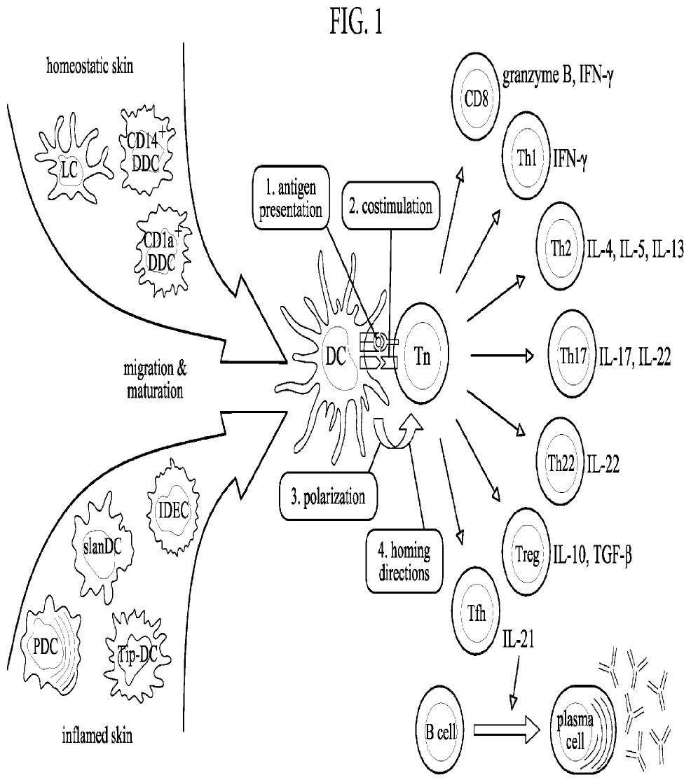

Overlapping peptide vaccines were originally conceptualized to induce immunomodulation from anaphylactic sensitization to tolerance without the vaccines themselves being able to trigger anaphylaxis. The rationale is to present the dendritic cells at the left-hand side of FIG. 1 with epitopes capable of inducing immunomodulation from antibody production (Tfh in FIG. 1) to tolerance (Treg) without provoking IgE cross-linking and mast cell degranulation. In overlapping peptide vaccine immunotherapy the peptides are formulated to contain all the 9 AA sequences of either the intact parent or alternatively of recognized target epitopes that might fit the antigen presenting grooves of the recipient's dendritic cell MHC class II molecules (Arnold PY, La Gruta NL, Miller T, Vignali KM, Adams PS, Woodland DL and Vignali DAA: The majority of immunogenic epitopes generate CD4+ T cells that are dependent on MHC class II-bound peptide-flanking residues. J Immunol. 2002 Jul. 15;169(2):739-49. doi: 10.4049/jimmunol. 169.2.739). When the disease states to be treated include IgE-mediated anaphylaxis the vaccines must be free of either homologous (Kane PM, Holowka D & Baird B: Cross-linking of IgE-Receptor Complexes by Rigid Bivalent Antigens >200 A in Length Triggers Cellular Degranulation. J Cell Biol 1988;107:969-980) or heterologous (Göbl C et al: Flexible IgE epitope containing domains of Phl p 5 cause high allergenic activity. J Allergy Clin Immunol. 2017 October; 140(4):1187-1191. doi: 10.1016/j.jaci.2017.05.005) bivalency that could cross-link IgE molecules on mast cells and trigger degranulation.

When available, overlapping peptide vaccines can be alternatives to the complete proteins from which their sequences are derived, for many modalities of allergen immunotherapy. They can be preferred for immunomodulation from allergic sensitization to tolerance to antigens for which exposure to the intact protein could induce anaphylaxis. Overlapping peptide vaccines avoid this adverse effect by presenting the relevant epitopes of the intact protein allergen but in short enough segments to be unable to cross-link IgE receptors on mast cells (Huang Y-F, Liu H, Xiong X, Chen Y and Tan W: Nanoparticle-mediated IgE-Receptor Aggregation and Signaling in RBL Mast Cells. J Am Chem Soc. 2009 December 2; 131(47): 17328-17334. doi: 10.1021/ja907125t). However, even at the increased doses that can be safely administered with vaccines that are not capable of cross-linking IgE on mast cells, peptide vaccines have not been universally effective.

An overlapping peptide vaccine for cat allergy that failed to achieve clinical trial objectives (ToleroMune-cat, Circassia since licensed to Adiga Lifesciences) illustrates two liabilities commonly encountered in previous overlapping peptide vaccines that are addressed by the present invention.

The first is inability to include peptides containing all epitopes of the target protein because of lack of solubility. Peptides vary in their natural solubility patterns as a function of their amino acid sequences. Some of the overlapping peptides made to cover the entire amino acid sequence of the protein that was the target of that vaccine were insoluble in its intended vehicle and for this reason left out of the formulation used in the unsuccessful clinical trial.

Nucleophile/Electrophile/Silylating Reagents (ACS Catalog 2020.10.16.9594, Publication Date: July 31. 2020) are a class of reagents capable of coupling solubility-modifying side chains to the N-terminal amino and C-terminal carboxyl residues of peptides and in theory could be used, by coupling to hydrophilic or hydrophobic side chains, to render complete sets of informationally significant overlapping peptide vaccines either soluble or insoluble in water and in the latter case soluble in one or more of the three pharmaceutically acceptable water-miscible solvents (ethanol, acetonitrile and dimethylsulfoxide (DMSO)). Coupling with hydrophilic “tails” of polyethylene glycol, called “PEGylation” for which one vendor's website is https://www.cd-bioparticles.com/support/polyethylene-glycol-peg-modification.html, can be done to impart water-solubility when coupled with reactive agents that bind with different classes of protein binding sites also including both free amino and free carboxyl residues.

However, Circassia chose to abandon a vaccine in which it had invested tens of millions of dollars likely because then-known methods of peptide solubility modification by coupling to solubility-modifying “tails” including those described above and others originally developed to modify the solubility of proteins as discussed in Coifman RE & Yang CF: Novel allergy vaccine delivery system for poison ivy urushiol (PI) and Peanut (PN). Poster 1019 presented to World Allergy Organization Symposium on Immunotherapy and Biologics, Chicago IL Dec. 13, 2013. Recipient of WAO Top Abstract Award, are insufficiently selective for C- and N-terminal binding. Many such reactions can be conducted under conditions that are relatively selective for C- and N-terminal binding but coupling with a sufficient molar excess to assure achievement of target solubility goals will inevitably result in sufficient coupling to exposed amino and carboxyl groups of arginine, histidine, lysine and aspartic and glutamic acids within the overlapping peptide chains blocking clinically relevant epitopes to alter antigenicity and either blunt or completely block any immunomodulatory effect. This adverse effect of solubility modification by coupling could be completely avoided and all the informationally significant AA sequences of ToleroMune-cat rendered water soluble without alteration of their antigenicity by sandwiching their informationally significant AA manufacture between sufficiently long informationally insignificant sequences of hydrophilic AA's according to the novel methods of the present invention, which were not contemplated at the time.

The second liability illustrated by ToleroMune-cat is less efficient delivery to the immunomodulatory mechanism of the immune system than could be expected with VDBP.

In the terminology of physics, the system shown in FIG. 1 has inertia and takes force to change direction. It is also digital. As the inventors learned from their human proof-of-concept experience with poison ivy, increasing treatment dose increased the percentage of patients who responded to treatment but the response was in almost all cases all or none. A fraction of a more effective dose did not produce a fractional partial response, it simply flipped the switch from sensitization to tolerance in a smaller fraction of treated patients. The other lesson from the inventors' experience with poison ivy is that TDBP (or, for vaccines, VDBP) is a force multiplier. The inventors' poison ivy urushiol vaccine was safe and effective when administered by TDBP/VDBP whereas 100 years of efforts by others with the same urushiols but administered by other means failed to flip the switch from sensitization to tolerance in a statistically significant fraction of treated individuals. Solubility modification of the informationally active sequences of overlapping peptide vaccines to permit delivery by TDBP/VDBP could be expected to increase the immunomodulatory “force” of TDBP/VDBP formulations from either sensitization to tolerance or from tolerance or naivete to protective sensitization. As with the above example of coupling to make the water insoluble peptides of ToleroMune-cat water soluble, however, this cannot be accomplished with previously reported methods of solubility modification by coupling because of insufficient specificity. Also as in the above example and as an example embodiment of the present invention, informationally significant peptide vaccines can be given the solubility properties needed for TDBP/VDBP without their information content being disrupted by the unintended binding of solubility-modifying coupling reagents to exposed amino and carboxyl residues on arginine, histidine, lysine and aspartic and glutamic acids within those peptide chains by sandwiching those informationally significant peptides between informationally insignificant strings of hydrophobic AAs of sufficient length to render them insoluble in water and soluble in the water-miscible solvents most appropriate for their intended routes of administration. These could vary by individual application but would generally be ethanol or acetonitrile for injection and DMSO for topical application. These informationally insignificant AA sequences could be programmed to be manufactured both before and after the informationally significant AA sequences to be given by TDBP/VDBP to trigger therapeutic immunomodulation in either direction.

Random human donor serum may contain IgE antibody against randomly generated peptide sequences (Krause T, et al: IgE Epitope Profiling for Allergy Diagnosis and Therapy—Parallel Analysis of a Multitude of Potential Linear Epitopes Using a High Throughput Screening Platform. Front. Immunol., 30 September. 2020|https://doi.org/10.3389/fimmu.2020.565243). The total length of the solubility-modified peptides will be greater than the 5 nM estimated minimum distance between epitopes needed for IgE cross-linking and mediator release (Knol EF: Requirements for effective IgE cross-linking on mast cells and basophils. Mol. Nutr. Food Res. 2006, 50, 620-624 DOI 10.1002/mnfr.200500272). The risk of accidentally presenting epitopes capable of cross-linking will be greatest in patients being treated for IgE-mediated allergic diseases who have already demonstrated their ability to mount IgE-mediated allergic mediator release reactions. It can be minimized for all patients if the entire lengths of all solubility-modifying AA sequences used as solubility modifiers in the same overlapping peptide vaccine are repeated insertions of the same AA. If folding of the AA tails is needed to control either solubility or viscosity, efforts should be made to use only a single hydrophobic AA for all residues separated by more than 5 nM from the far end measured along the length of the peptide chain. The 3D structure of almost any peptide with 5 to 50 AAs can be modeled with resources such as RPBS PEP-FOLD, (on-line at https://mobyle.robs.univ-pa aris-diderot.fr/cgi-bin/portal.pvitforms::PEP-FOLD3), allowing estimates of solubility and in silico comparison of the suitability of different individual hydrophobic AAs. Solubility-modifying sequences of smaller molecular size hydrophobic acids glycine and alanine may result in lower viscosity, more rapid dilution and smaller resulting particle size than similar sequences of larger and bulker hydrophobic AAs.

The process of solid phase peptide synthesis was first reported in by Merrifield in 1963 (Merrifield RB (1963). “Solid Phase Peptide Synthesis. I. The Synthesis of a Tetrapeptide”. J. Am. Chem. Soc. 85 (14): 2149-2154. doi: 10.1021/ja00897a025). First an amino acid with its amino terminal protected is coupled to a polystyrene resin. The amine is then deprotected, coupled to the free carboxyl end of the second amino acid. This cycle repeats until the desired sequence has been synthesized. Since its original report, the process has been improved and automated so that today an operator can simply program an amino acid sequence, load an automated Solid Phase Protein Synthesis (SPPS) machine with the necessary reagents, push “Start” and wait for the programmed peptide to be made. In the present invention, the traditional use of SPPS to make informationally significant AA sequences is supplemented by its use to insert informationally insignificant AA sequences both before (at the C-terminal end) and after (at the N-terminal end) of an informationally significant AA sequence for the purpose of conferring the solubility properties needed to equip the resulting product for tissue delivery by precipitating (TDBP) and if its intended use is as a vaccine, for VDBP.

Sensitization of recipients of vaccines against infectious viruses to viral epitopes that conformationally mimic receptors or their agonists important to physiological homeostasis can induce pathological anti-idiotype antibodies. Such antibodies to the angiotensin II receptor to which the SARS COVID-19 virus binds as its mechanism of cell entry have been blamed for many late or persistent adverse reactions to COVID-19 mRNA and viral vector vaccines (Murphy W J and Longo D L: , M. D: A Possible Role for Anti-idiotype Antibodies in SARS-CoV-2 Infection and Vaccination. New England J Medicine. doi.org/10.1056/NEJMcibr2113694). These epitopes are technically difficult to edit out of mRNA and viral vector vaccines but much easier to eliminate from the inventors' proposed TDBP/VDBP vaccines, making it easier to make safer vaccines against such pathogens using TDBP/VBP technology. For viruses that infect by way of the nasal mucosa, TDBP/VDBP vaccines also offer the advantage of immunization by way of the nasal mucosa resulting in local cell mediated immunity as well as systemic immunity.

The present inventors injected into tissue of a recipient, a water-insoluble antigen in a pharmaceutically acceptable water-miscible solvent (such as ethanol) that carried antigen with it as it spread from the injection site. As the small volume of injected ethanol was diluted by the water content of the recipient tissue the urushiol became insoluble and precipitated. The more rapid the dilution the larger the number and smaller the size of the resulting particles. Particles in the 0.5 to 5 micron size range are efficiently taken up by naive dendritic antigen-presenting cells (APC's) by macropinocytosis (Xiang). What the inventors have achieved was a balance between injected volume, viscosity and access to tissue water to achieve a rate of dilution yielding hundreds of thousands to millions of particles spanning a size range for efficient uptake by naive APC's. The inventors chose a tissue (skeletal muscle) in which the primary evolutionary role of the immune system is the maintenance of tolerance to self. The lineages of dendritic cell populations present in muscle are expected by the inventors to be primarily tolerogenic and the cytokine milieu of the lymph nodes to which those dendritic cells bring antigen to present to naive T cells to be similarly biased toward immunomodulation from sensitization to tolerance.

The present application is directed to, inter alia, methods by which peptides in general as well as the subset of peptides containing epitopes of allergens for which one wants to induce immunomodulation to either tolerance or sensitization, in particular, can be deposited by precipitation in tissues in which the immune system is evolutionarily predisposed to the induction of either tolerance or protective sensitization, to take advantage of the inventors' particulate form of delivery.

The binding grooves of the APC MHC class II proteins that present antigens to naive T-cells for immunization or immunomodulation hold peptides or segments of peptides 9 amino acids in length. Singly amino acid-shifted 9 amino acid (or optionally longer) overlapping peptides of the amino acid sequences of proteins to which one wants to modulate the immune response should encompass all immunologically relevant T-cell epitopes based on current understanding of the underlying cellular and molecular mechanisms.

To become capable of TDBP/VDBP, however, the peptides must be sufficiently hydrophobic to be insoluble in water and at the same time highly soluble (enough to dissolve therapeutic doses in small fraction of a milliliter volumes) in one or more of the pharmaceutically acceptable solvents, including but not limited to, ethanol, acetonitrile and DMSO. The traditional method of protein or peptide solubility modification, well known to those skilled in the art, is coupling to end-chains or side-chains with solubility-modifying properties spanning a sufficiently larger area of molecular interface than that of the native protein or peptide to determine its solubility pattern. For epitope-sized peptides such coupling agents have the disadvantage that a certain fraction of coupling reagents directed at the reactive C- and N-terminal amino and carboxyl ends of the peptides whose solubility they are intended to modify will instead bind to exposed amino and carboxyl residues on charged amino acids arginine, histidine, lysine and aspartic and glutamic acids within those peptide chains, modifying their antigenicity and ability to produce the intended immunomodulation upon TDBP/VDBP.

In the present invention, the inventors overcome this obstacle by not starting with peptides that replicate the overlapping amino acid sequences of the antigenic protein to which the inventors want to modify the immune response and then attempting to modify their solubility. Instead, in the present invention, peptides are synthesized that sandwich overlapping amino acid sequences from the target antigenic protein between strings of hydrophobic amino acids pre-programmed in place to provide the requisite solubility profile for TDBP (applicable to both antigenic peptides for VDBP and non-immunologic applications that fall within TDBP but not VDBP) without exposing side chain amino and carboxyl groups to reactions that could result in any form of inactivation.

For the safe induction of immunological tolerance in allergic diseases for which the spectrum of manifestations includes anaphylaxis, it is important that the synthesized peptides that comprise the vaccine not be capable of bridging IgE receptors on mast cells (Huang Y-F, Liu H. Xiong X, Chen Y and Tan W: Nanoparticle-mediated IgE-Receptor Aggregation and Signaling in RBL Mast Cells. J Am Chem Soc. 2009 Dec. 2; 131(47): 17328-17334. doi: 10.1021/ja907125t). The strings of hydrophobic amino acids needed to confer the solubility properties for TDBP will make those segments rigid and if long enough potentially capable of bridging mast cell-bound IgE molecules and triggering anaphylaxis in the aqueous environment of the tissues into which they are precipitated, IF they inadvertently contain any second epitope to which recipient mast cells might also contain IgE. The likelihood that the hydrophobic amino acid sequences added to give the vaccines appropriate solubility might inadvertently contain epitopes to which the recipient happens to have IgE could be minimized by programming each such chain to be repeated monomers of the same hydrophobic amino acid. These single amino acid peptide chains will be sufficiently non-physiologic to have an extremely low likelihood of having been previously encountered by the vaccine recipient which could lead to mast cell presence of reactive IgE.

Overlapping peptide vaccines will contain linear but not conformational epitopes. However, Berglund et al point out that most (and suggest that all) discontinuous (I.e., conformational) epitopes are composed of short linear epitope sequences forming a binding region for the antibody (Berglund et al.). A complete set of overlapping peptide vaccines will encompass all such short linear epitopes. When their exact sequence and location are known, extraneous peptides may be left out of the vaccine.

The task of the present invention has three components: 1) Formulate vaccines for effective MHC type II presentation. 2) Give them the requisite solubility properties for VDBP without compromising their ability to perform task #1. 3) Find ways to deliver them to dendritic cells of lineages predisposed to the intended direction of therapeutic immunomodulation, for presentation to naive T cells in cytokine environments predisposed to immunomodulation in the same direction.

### Choice of Epitopes to Omit for Reasons of Safety

Immunization with epitopes that are foreign (and therefore antigenic) but that bind to autogenous receptors involved in the control of any physiologic process can induce the formation of anti-idiotype antibodies able to confound numerous physiologic processes and potentially responsible for many adverse immunologic sequelae of both COVID-19 infection and COVID-19 vaccines (Kim YC, Jarrahian C, Zehrung D, Mitragotri S and Prausnitz MR: Delivery Systems for Intradermal Vaccination In Teunissen MBM, editor, Intradermal Immunization, Current Topics in Microbiology and Immunology Volume 351, pp. 76-112, Springer, Heidelberg, ISSN 0070-217X, ISBN 978-3-642-23689-1 e-ISBN978-3-642-23690-7 DOI 10.1007/978-3-642-23690-7). Such epitopes are technically very difficult to exclude from nucleic acid or viral vector vaccines but much easier to exclude from overlapping peptide vaccines: Peptides containing those epitopes can simply be omitted from the sets manufactured for vaccine use.

### Choice of Target Tissues

Immunomodulation from sensitization to tolerance: Skeletal muscle was the recipient tissue for successful tolerance induction to poison ivy by injection in pharmaceutically acceptable volumes of ethanol (Coifman R E, Yang C F, Tolerance to poison ivy following vaccine delivery by precipitation, Annals of Allergy, Asthma and Immunology 2019 (Mar); 122:331-33). Skeletal muscle is a tissue in which the primary evolutionary role of the immune system is to maintain tolerance to self and should therefore be primarily populated by tolerogenic lineages of dendritic cells and have a tolerogenic cytokine environment. The tissue contemplated by the present invention is not limited to skeletal muscle, however.

Immunomodulation from tolerance in oncology and naiveté in infectious disease to protective sensitization: The dermis and the lining membranes of the nose are tissues whose primary evolutionary role is protection against infection. These tissues should be primarily populated by sensitizing lineages of dendritic cells and have an allergenic cytokine environment. Topically applied immunizing agents dissolved in DMSO will be carried through the essentially water-free epidermis and into the dermis as the DMSO diffuses inward across the skin barrier. TDBP vaccines dissolved in any of the 3 solvents listed below can be delivered to the dermis using devices designed for general dermal vaccine delivery (Kim YC, Jarrahian C, Zehrung D, Mitragotri S and Prausnitz MR: Delivery Systems for Intradermal Vaccination In Teunissen MBM, editor, Intradermal Immunization, Current Topics in Microbiology and Immunology Volume 351, pp. 76-112, Springer, Heidelberg, ISSN 0070-217X, ISBN 978-3-642-23689-1 e-ISBN978-3-642-23690-7 DOI 10.1007/978-3-642-23690-7).

Accordingly, multiple target tissues are contemplated within the scope of the present invention. Non allergy-immunology applications may require consideration of other target tissues.

### Choice of Solvent and Target Tissue-Specific Methods of Administration

Ethanol, acetonitrile and dimethylsulfoxide (DMSO) are water-miscible solvents of which sub-milliliter doses are pharmaceutically acceptable for administration to multiple potential target tissues by injection. Ethanol and acetonitrile are low viscosity solvents and small volumes should support peptide solutions of low enough viscosity to precipitate particles in the 0.5 to 5 micron size range for macropinocytosis by migrating naive dendritic APC's (Xiang SD, Scholzen A, Minigo G, et al. Pathogen recognition and development of particulate vaccines: does size matter? Methods. 2006;40:1e9). Particles in the 0.5 to 5 micron size range for macropinocytosis were proven superior to soluble forms of the same antigens for immunomodulation both from sensitization to tolerance (Neimert-Andersson T, Thunberg S, Swedin L, Wiedermann U, Jacobsson-Elunan G, Dahlen S.-E. Scheynius A, Gronlund H, van Hage M and Gafvelin G: Carbohydrate-based particles reduce allergic inflammation in a mouse model for cat allergy. Allergy 2008:63: 518-526) and from naiveté to sensitization (Kovacsovics-Bankowski M, Clark K, Benacerraf B, Rock K L. Efficient major histocompatibility complex class I presentation of exogenous antigen upon phagocytosis by macrophages. Proc Nat Acad Sci USA 1993;90:4942-4946).

The combination of ethanol as a solvent and skeletal muscle as a target tissue has proven effective for immunomodulation from sensitization to tolerance in allergy to poison ivy, with 0.15 ml maximum volumes of one or more physically closely spaced individual injections. Multiple injections were sometimes required to achieve target treatment dose within the inventors' arbitrarily chosen maximum ethanol volume of 0.15 ml. When multiple injections were needed they were given in close proximity in the same target tissue to maximize likelihood that dendritic cells scavenging precipitated antigen would present it at the same lymph node or set of nodes. Multiple injections may be needed for overlapping peptide vaccines to keep vaccine viscosity low enough to achieve a rate of solvent dilution that precipitates particles in the 0.5 to 5 micron size range for dendritic cell uptake by macropinocytosis.

Substitution of pharmaceutically acceptable volumes of low viscosity acetonitrile for ethanol as a vaccine vehicle for injection into skeletal muscle may reduce vaccine viscosity, increase rate of solvent dilution, and reduce precipitated particle size in applications in which ethanol yields particles that are too large.

If viscosity of vaccines intended for injection in any solvent becomes an issue, it can be addressed by dividing the same total treatment dose into multiple injections of less concentrated solutions. DMSO is more viscous than either ethanol or acetonitrile and if injected may not be diluted rapidly enough to precipitate particles of vaccine in the size range for macropinocytosis.

The viscosity of DMSO as a single component solvent may be too high to achieve effective particle size distribution for VDBP on injection into any target tissue. Combinations of DMSO with either ethanol or acetonitrile may allow effective VDBP for vaccines that are not adequately soluble in ethanol or acetonitrile alone.

Topically applied vaccines in DMSO may be capable of effective vaccine particle size delivery by a mechanism independent of its viscosity, because of its ability to penetrate and carry dissolved solute across biological phospholipid membranes including intact skin. Topically applied DMSO will carry dissolved vaccine with it as it traverses the phospholipid membranes of either skin or nasal mucosa. A wave of topically applied

DMSO will diffuse across both cellular and tissue phospholipid membranes carrying with it dissolved solute. Movement of the solute front will be slowed by what is essentially tissue chromatography as the solvent is also diluted by tissue water. Particles of vaccine will precipitate as micro-environmental DMSO levels fall because of the combination of dilution by tissue water and chromatographic slowing of the advancing front of solute behind the advancing front of solvent. Vaccine carried across phospholipid membrane by the diffusion of topically applied DMSO may become insoluble and precipitate at a more rapid rate than if the same vaccine was delivered by injection. The reason is that viscosity is a property of molecular interactions of like with like while molecules of DMSO that diffuse following topical application are in a microenvironment in which the molecules with which they interact are predominantly unlike themselves. In either case the resulting particle size distribution is a function of rate of solvent dilution with water at the molecular level. For a viscous solvent such as DMSO this rate is simply slower when each molecule of viscous solvent is surrounded by other molecules of the same viscous solvent than when it is surrounded by molecules of the tissue into which it is diffusing and the available water content that tissue contains.

The lining membranes of the nasal mucosa are sufficiently thinner than skin that they may allow effective penetration by vaccines dissolved in ethanol or acetonitrile as nose sprays, or by vaccines in DMSO applied as any of drops, sprayed droplets or painted on with swabs or other topical applicators.

Thus, it is contemplated that several appropriate pharmaceutically acceptable solvents may be used in accordance with the present invention, including combinations of certain solvents taking into account the factors indicated herein.

The size and location of precipitated particles of VDBP vaccines formulated for any form and target tissue of administration can be traced by administration by the same route to the same target tissue of a mouse, euthanization and electron microscopy (E/M) of the area surrounding the administration site. The glutaraldehyde fixative used for E/M will cross-link peptides in place (Mascorro, et al., Migneault I, Dartiguenave C, Bertrand M J & Waldron KC: Glutaraldehyde: behavior in aqueous solution, reaction with proteins, and application to enzyme crosslinking. BioTechniques 37:790-802, November 2004), producing a clearly demarcated homogeneous mass that should be easily identifiable and measurable. If particle size is above target, viscosity can be reduced by reducing total peptide concentration of which viscosity is an exponential function (Gonçalves, A.D., Alexander, C., Roberts, C.J. et al. 2016) The effect of protein concentration on the viscosity of a recombinant albumin solution formulation. RSC Advances, 6. pp. 15143-15154.). Viscosity may be further reduced by altering the composition of the solubility-modifying hydrophobic AA end-chains to reduce intermolecular interactions. Solubility-modifying chains of physically smaller hydrophobic AAs glycine and alanine may have less of a stearic contribution to viscosity than chains larger and bulkier AAs.

Tissues in which the primary evolutionary role of the immune system is protection against invading pathogens are more likely to be populated by sensitizing lineages of dendritic cells and have lymph node cytokine milieus that favor protective sensitization. These include the dermis and the lining of the nasal mucosa. Vaccines in DMSO applied topically to either skin or nasal mucosa may achieve effective vaccine precipitation rates to produce particles appropriately sized for macropinocytosis. Vaccines dissolved in any of the three solvents may be delivered to the dermis by injection or with any of a number of existing intradermal vaccine injection technologies (Kim Y C, Jarrahian C, Zehrung D, Mitragotri S and Prausnitz M R: Delivery Systems for Intradermal Vaccination In Teunissen MBM, editor, Intradermal Immunization, Current Topics in Microbiology and Immunology Volume 351, pp. 76-112, Springer, Heidelberg, ISSN 0070-217X, ISBN 978-3-642-23689-1 e-ISBN978-3-642-23690-7 DOI 10.1007/978-3-642-23690-7).

The vaccine delivery processes of VDBP is not referred to as “atraumatic,” as exposure to any of the three solvents will cause some degree of transient chemical shock. That shock may be sufficient to enable vaccines in ethanol or acetonitrile applied by spraying in a metered dose mist to penetrate and precipitate within the tissue of the nasal mucosa. Vaccines dissolved in more viscous DMSO can be similarly applied by brushing or as drops. While none of the cited methods of TDBP are completely atraumatic, however, TDBP is far less traumatic than any other known or (to the knowledge of the present inventors proposed) to populate a volume of a target tissue with an administered exogenous substance in the form of thousands to millions of sub-micron to multi-micron sized particles.

The nasal mucosa is a particularly attractive site for TDBP/VDBP for immunization against infectious diseases for which the nasal mucosa is the primary portal of entry, such as COVID-19. For such diseases the induction of local cell-mediated immunity can reduce both infection and transmission. For COVID-19 there are good animal models in which some (van Doremalen N et al: Intranasal ChAdOx1 nCOV-19/AZD1222 vaccination reduces shedding of SARS-CoV-2 D614G in rhesus macaques. bioRxiv preprint doi: https://doi.org/10. 1101/2021.01.09.426058.) but not all (Furuyama W et al: Rapid Protection from COVID-19 in Nonhuman Primates Vaccinated Intramuscularly but Not Intranasally with a Single Dose of a Vesicular Stomatitis Virus-Based Vaccine. https://journals.asm.org/journal/mbio Jan/Feb 2022; 13 (issue 1) e03379-21) intranasally applied vaccines have induced protective immunity.

The presence of inflammation induces different populations of dendritic cells, as illustrated in the lower left hand corner of FIG. 1. The deliberate induction of a cell-mediated allergic reaction in the same site either prior to or concurrent with overlapping peptide VDBP will increase influx of dendritic cells of lineages associated with allergic sensitization and may further bias the response toward protective sensitization. Sensitizers appropriate for this use should be universal, so that all or nearly all recipients will respond. They should be non-natural, so that no recipients would be naturally sensitized and at unrecognized risk for more severe reactions because of prior sensitization to doses determined to be safe and effective in previously unsensitized recipients. Use of the historical standard sensitizer of this class, dinitrochlorobenzene (DNCB) is controversial because it fails the Ames test for mutagenicity (Happle R: The Potential Hazards of Dinitrochlorobenzene. Arch Dermatol (1985) 121:330-1). However, its clinical efficacy as an inducer of protective sensitization against the tumor-inducing virus of verruca vulgaris raises questions about whether its mutagenicity on the Ames in fact indicates a risk of carcinogenicity in human use. Alternative Ames test-negative universal sensitizers have been proposed (Happle). The added efficacy (increase above baseline efficacy of the same VDBP protocol in the absence of a same site induced cell mediated allergic reaction) can be studied in the above animal models. Variables that may impact the added efficacy of an induced cell mediated reaction include timing with respect to vaccine delivery and intensity of the induced cell mediated reaction.

### Choice of Hydrophobic Amino Acids to Use for Solubility Modification

Non-limiting example peptide sequences are composed exclusively of single amino acid or mixed polymers of alanine, isoleucine, leucine, methionine, phenylalanine, proline, tryptophan, valine, threonine and tyrosine will be soluble in any of ethanol, acetonitrile and DMSO and insoluble in water. Proteins composed of exclusively of single amino acid or mixed polymers of the same group of amino acids plus serine will be insoluble in water but soluble in ethanol.

Length of hydrophobic amino acid C- and N-terminal “tails” needed to achieve solubility targets: Non-limiting examples may require empirical determination for individual applications.

### Non-Limiting List of Potential Applications of TDBP with Solubility-Engineered Peptides

a. Non-Limiting Example Candidate Allergens for Immunomodulation from Sensitization to Tolerance
Anaphylactic (IgE-mediated) food allergy,
Food-allergic dermatologic and gastrointestinal diseases,
Anaphylactic allergy to antigens transmitted by insect bites and stings,
Allergy to heterologous proteins in tissue transplants,
Common environmental inhalant aeroallergens,
Macromolecular protein occupational allergens.
Tissue protein antigens of autoimmune diseases.
   b. Non-Limiting Example Candidate Allergens for Immunomodulation from Tolerance (in Cancer and Some Infectious Diseases) or Naiveté (in Other Infectious Diseases) to Protective Sensitization
Every cancer patient's individual tumor-specific antigens.
Epidemic/pandemic viral infections including COVID-19, other epidemic coronaviruses and other viruses including Ebola, Zika, influenza, insect-born viral encephalitides, others.
Malaria and similar parasitic diseases.
Tuberculosis and other treatment-resistant mycobacterial infections.
Tick-borne rickettsial diseases.

### Immunomodulation from Tolerance to Sensitization

Examples include all cancers and also non-malignant tumors.

### Examples of Immunomodulation from Naiveté to Protective Sensitization Against the Pathogens Causing

Epidemiologically significant bacterial diseases such as tuberculosis, streptococcal and vibrio vulnificus necrotizing fasciitis, clostridium difficile
Epidemiologically significant parasitic diseases such as malaria
Epidemiologically significant &/or difficult to manage rickettsial diseases
Epidemiologically significant viral diseases including Ebola, Zika, COVID-19.COVID-19 is a particularly favorable candidate for protective immunization by TDBP to the nasal mucosa as that would induce local protective immunity at the most common site of infection as well as systemic immunity COVID-19.

### Choice of Epitopes to Exclude from ILIT-Adapted Overlapping Peptide Vaccines

When the complete AA sequence of all clinically relevant epitopes is known, overlapping peptides that do not contain AA sequences of relevant epitopes should be excluded from the vaccine. Reducing irrelevant peptide content will usually reduce viscosity and facilitate ability to achieve rates of solvent dilution yielding particles in the target size range of 0.5 to 5 microns. When the objective is immunomodulation from naiveté or tolerance to sensitization (in cancer or infectious diseases), exclusion of epitopes replicating agonists or antagonists of human metabolic processes will reduce or eliminate the risk of anti-idiotypic antibody-mediated adverse effects (Murphy W J and Longo D L: , M. D: A Possible Role for Anti-idiotype Antibodies in SARS-CoV-2 Infection and Vaccination. New England J Medicine. doi.org/10.1056/NEJMcibr2113694).

FIG. 1 is a graphic representation of the function of the adaptive immune system in the skin, reproduced from Teunissen on intradermal immunization. It is a cartoon of how immunomodulation takes place. The left of FIG. 1 shows dendritic cells of which different populations normally inhabit both the epidermis and the dermis. These recognize and take up molecules or particles of an antigen which they then present to naive T lymphocytes which then mature along one of what are shown here as seven different pathways. The location is the cortex of a lymph node. Dendritic cells (DC's) from either homeostatic or inflamed tissue present antigenic epitopes in their MHC Class Il molecular grooves, to naive T lymphocytes (Tn). These can tolerize by maturing into Treg cells, sensitize by maturing along any of the other illustrated tracks, or immunomodulate by outnumbering &/or displacing already present T-cells of one type with those of another. The cellular and molecular mechanisms are the same for immunomodulation from sensitization to tolerance in allergy and from tolerance in cancer and naiveté or tolerance in infectious diseases to protective sensitization. The switches are simply flipped in different directions (O'mahony). In the terminology of physics, the system has inertia and it takes force to change its direction. The present inventors' successful change of direction with poison ivy VDBP suggests that VDBP gives an antigen a force amplifier that might help it flip other previously flip-resistant switches of immunological responsiveness, as well. The urushiols of poison oak and poison ivy naturally have the solubility properties needed for VDBP: Insolubility in water combined with sufficient solubility in at least one of the three pharmaceutically acceptable water miscible solvents (ethanol, acetonitrile and DMSO) for a treatment dose to be dissolved in a pharmaceutically acceptable volume of the solvent.

Both the direction and the intensity of the lymphocyte response are determined by which population of dendritic cells picks up and delivers the antigen to the naive lymphocytes at the right middle of the figure, and the cytokine milieu in which they do it.

The dendritic cells shown at the upper left are those present in skin under normal or homeostatic conditions. Those at lower left are the populations generated or recruited in response to inflammation. The physiology of the primary function of the immune system in the skin is that if an infectious organism breaks through the physical barrier or the non-living outer layers of the skin, its presence is recognized by the innate immune system which elicits a local inflammatory reaction. This provokes the recruitment and generation of the new populations of dendritic cells shown at lower left and also alters the local cytokine milieu. The function of those new populations of dendritic cells and altered cytokine milieu is to facilitate a protective immune response to the infecting organism that will be recalled to defend the host if the provoking infection persists or any time that infecting organism is encountered again.

When target epitope sequences are known, the overlapping peptide sets to be used as vaccines need only contain all of the 9 AA sequences (the capacity of the MHC II binding groove (Arnold, et al.)) included in those epitopes. When all relevant epitope sequences and locations are not known, use of longer overlapping peptides will reduce the mass of extraneous material that must be included for a vaccine to contain all potentially relevant 9 AA sequences. A single peptide 18 AAs long will contain 10 unique 9-AA sequences, for example, while it would require 10 separate peptides each 9 AAs long in addition to their solubility-modifying “tails” in length to provide the same epitope diversity. Eliminating extraneous content will reduce vaccine viscosity; higher viscosity in injected vaccines will reduce the rate of solvent dilution and risk shifting particle size distribution out of the 0.5 to 5 micron range needed for dendritic cell uptake by macropinocytosis.

For MHC Class II binding and antigen presentation of epitopes in sequences more than 9 AAs long, tighter MHC class II protein binding resulting in a greater efficacy is achieved if the adjacent AA at each end of the 9 AA sequence presented for immunological recognition is hydrophilic (Stawikowski et al.). The AAs adjacent to internal 9 AA epitope candidates in overlapping peptides more than 9 AAs in length (in addition to their C-terminal and N-terminal hydrophobic AA tails) cannot be controlled. However, a hydrophobic AA can be inserted at each end of the overlapping peptide sequence of the parent protein, between it and the solubility-modifying strings of hydrophobic AA's at either side.

In summary, the inventors learned from poison ivy that precipitation of a water-insoluble antigen as a water-miscible solvent in which it's administered is diluted by the water content of a recipient tissue, can be a potent and efficient way to feed antigen to APC's to induce therapeutic immunomodulation. Methods are outlined herein to exploit the same boost to the efficiency with which clinically relevant epitopes of protein antigens can be presented for therapeutic immunomodulation to both from sensitization to tolerance in allergic diseases and from tolerance in cancer and naiveté or tolerance in infectious diseases to protective sensitization. Potential obstacles to the achievement of these goals are reviewed, and methods are provided to track the efficacy of vaccine delivery to its intended location and in the effective range of particle size.

Non-limiting example embodiments of the present invention are directed to the production of vaccines for VDBP, for which modified peptides must be insoluble in water but with doses that are soluble in pharmaceutically acceptable volumes of one or more of the water-miscible solvents ethanol, acetonitrile and DMSO. According to non-limiting example embodiments, solubility modification of peptides by manufacturing them with strings of solubility-directing individual amino acids could be used to increase, rather than decrease, solubility in water if it was done with amino acids that are hydrophilic rather than hydrophobic. Solubility modification by sandwiching the immunologically active overlapping peptide sequences between solubility-modifying amino acid sequences that were hydrophilic rather than hydrophobic, could have resolved the first liability of Circassia ToleroMune-cat by making those of its unmodified overlapping peptides that were originally insoluble in its intended vehicle (water) water-soluble.

The history of ToleroMune-cat illustrates the non-obviousness of the present invention. The ToleroMune-cat invention failed to meet its efficacy endpoint in a pivotal clinical trial. Polyethylene glycol as a commercially available hydrophilic tail sold with reactive coupling groups that bind to both free amino and free carboxyl groups. If the developers of ToleroMune-cal thought of solubility modification they decided against it because the high likelihood that the same coupling reagents would bind to a sufficient fraction of the exposed amino and carboxyl residues on intra-chain arginine, histidine, lysine and aspartic and glutamic acids within the overlapping peptide chains to alter their antigenicity and make them incapable of inducing their intended immunomodulation. This would address the first of the above-cited liabilities of that vaccine but not the second. However, there may be circumstances in which individual peptides or groups of peptides may be more able to perform their intended functions if modified to make them more, rather than less, soluble in water. Modification of the solubility of peptides in either direction by sandwiching their biologically active amino acid sequences between hydrophilic or hydrophobic amino acid chains in the programming of their manufacture are thus embodiments included within the scope of this invention.

As with the overlapping peptides from which Circassia made the ToleroMune-cat vaccine, cited above as an example, prior to the present invention efforts were usually not made to modify peptide solubility at all. The reason is that all previously available methods to do so, C-terminal and N-terminal coupling with chemically reactive coupling reagents attached to solubility-modifying tails, had a high enough frequency of coupling to exposed amino and carboxyl residues on arginine, histidine, lysine and aspartic and glutamic acids within the overlapping peptide chains to impede or totally block the intended biological or immunological activity. Extensive modification and reversal of the innate solubility patterns of either naturally hydrolyzed or already synthesized peptides could not be accomplished with prior methods of solubility modification by coupling reactive coupling reagents attached to solubility-modifying “tails” without impeding their immunological functions and potentially many of their non-immunological biological functions. Solid phase synthesis has heretofore been used to synthesize informationally significant AA sequences. It has not been previously employed to program the synthesis of informationally insignificant AA sequences at the ends of an informationally significant AA sequence for the exclusive purpose of modifying its solubility.

Embodiments of the present invention include methods, which include the programming of informationally insignificant solubility-modifying peptide chains at the ends of informationally significant AA sequences peptides to give them solubility properties of being insoluble in water but soluble in a pharmaceutically acceptable water-miscible solvent, the solubility properties for TDBP, without need for post-synthesis or post-hydrolysis coupling reactions.

Also included herein are informationally significant peptides made by the methods herein. Each informationally significant peptide may be programmed to include non-informational solubility-modifying peptide chains in amino acid sequences of the informationally significant peptide. According to example embodiments, after non-informational solubility-modifying peptide chains are added to the amino acid sequences of the informationally significant peptide, the informationally significant peptide has the solubility properties for TDBP, i.e. of being insoluble in water but soluble in a pharmaceutically acceptable water-miscible solvent.

The present invention includes the use of solid phase peptide synthesis for solubility modification of peptides by programming and inserting chains of relatively chemically and immunologically inert hydrophobic (to confer the solubility properties needed for TDBP/VDBP) or hydrophilic (to allow developers of water-soluble Vaccines such as Circassia's ToleroMune-cat) amino acid chains, to increase the likelihood of effectiveness by including ALL overlapping peptide sequences in their vaccine.

Non-limiting examples of the present invention include methods that include synthesizing solubility-modified sets of one or more peptides, which includes programming insertion of non-informational strings of hydrophobic amino acids at both C-terminal and N-terminal ends of peptides to be precipitated into non-liquid tissues by tissue deposition by precipitation. The non-informational strings of amino acids may be of uniform solubility. According to non-limiting embodiments, the methods include programming insertion of non-informational strings of amino acids of uniform solubility at both C-terminal and N-terminal ends of an informational amimo acid sequence, to allow the informational amino acid sequences to be administered by methods determined by the solubility properties of the non-informational amino acid sequences bonded to their C-terminal and N-terminal ends.

Further non-limiting examples of the present invention include methods of making solubility-modified peptides with the requisite solubility properties for tissue delivery by precipitation without extraneous binding and modification of antigenicity, which include
incorporating strings of hydrophobic amino acids as solubility modifiers at both C-terminal and N-terminal ends into epitope-containing segments of selected peptides using solid phase peptide synthesis to produced solubility-modified peptides. In non-limiting examples, the peptides are synthesized to be capable of precipitation within volumes of target tissues of a recipient, when a pharmaceutically acceptable solvent and the solubility modified peptides are administered to a target tissue of said recipient.

According to non-limiting examples of the present invention, the peptides are overlapping peptides of protein antigens, and the method further includes using solid phase peptide synthesis to incorporate strings of hydrophobic amino acids as solubility modifiers at both the C-terminal and N-terminal ends of into the epitope-containing segments of the overlapping peptides.

According to further non-limiting example embodiments, the protein antigens are causes of pathological sensitization.

According to non-limiting example embodiments, the peptides are selected and synthesized specifically for immunotherapy to produce immunomodulation from pathological sensitization to immunological tolerance.

The peptides according to the present invention may be exogenous antigens that are selected and synthesized specifically for treatment or prevention of allergic diseases. Example peptides may be endogenous antigens that are selected and synthesized specifically for treatment or prevention of autoimmune diseases.

According to example embodiments, the protein antigens are attributed to causes of pathological tolerance of a cancer or tumor and the protein antigens are selected and synthesized specifically for immunotherapy to produce immunomodulation from pathological tolerance of tumor antigens to protective sensitization.

According to non-limiting examples, the protein antigens are antigens of infectious diseases and the antigens are selected and synthesized specifically for immunotherapy to induce protective sensitization. Example antigens may be selected and synthesized specifically for immunotherapy to induce protective sensitization to diseases to which a recipient has not yet been exposed. According to other example embodiments, the antigens are selected and synthesized specifically for immunotherapy to induce protective sensitization to diseases to which a recipient has probably been exposed, but is not known to have been actively infected. According to further example embodiments, the antigens are selected and synthesized specifically for immunotherapy to induce protective sensitization to diseases to which a recipient is currently or has previously been infected. Non-limiting example diseases may include malaria or TB. According to other example embodiments, example diseases may include COVID-19.

In example embodiments, modified peptides are synthesized that sandwich overlapping amino acid sequences from a target antigenic protein between strings of hydrophobic amino acids pre-programmed in place to provide the requisite solubility profile for TDBP, without exposing side chain amino and carboxyl groups to reactions that could result in any form of inactivation.

Also included in the present invention are methods of making solubility-modified peptides with the requisite solubility properties for tissue delivery by precipitation without extraneous binding and modification of antigenicity. The present example methods include adding solubility-modifying non-epitope sequences of hydrophobic amino acids in a peptide assembly process at or past both ends of epitope-containing MHC-binding sequences, yielding TDBP/VDBP-compatible overlapping peptide sets with fully unblocked epitope amino acid sequences and with necessary solubility properties for VDBP, to produce solubility-modified peptides. According to these example embodiments, the solubility-modified peptides are synthesized to be capable of precipitation within volumes of target tissues of a recipient, when administered to said target tissue of said patient, as pharmaceutically acceptable solvents.

Further methods of the present invention include methods of making solubility-modified peptides with the requisite solubility properties for tissue delivery by precipitation without extraneous binding and modification of antigenicity, which include using solid phase peptide synthesis to sandwich immunologically active overlapping peptide sequences between solubility-modifying amino acid sequences that were hydrophilic to produce solubility modified peptides, in which the peptides are synthesized to be capable of precipitation within volumes of target tissues of a recipient, when a pharmaceutically acceptable solvent and said solubility modified peptides are administered to a target tissue of said recipient.

The present invention also includes methods of enhancing protective immunity in a subject, which methods include administering to a subject at least one peptide provided herein, including at least one peptide synthesized by any of the methods provided herein.

The present invention also includes solubility-modified peptides comprising insertion of strings of hydrophobic amino acids added by program at both C-terminal and N-terminal ends of peptides to be precipitated into non-liquid tissues by tissue deposition by precipitation. According to non-limiting example embodiments, the solubility modified peptides are synthesized by solid phase peptide synthesis by programming and inserting chains of relatively chemically and immunologically inert hydrophobic or hydrophilic amino acid chains to increase the likelihood of effectiveness by including overlapping peptide sequences in their vaccine.

Non-limiting example embodiments of the present invention include methods that include programming non-informational solubility-modifying peptide chains into amino acid sequences of informationally significant peptides to give them solubility properties of being insoluble in water but soluble in a pharmaceutically acceptable water-miscible solvent, without need for post-synthesis or post-hydrolysis coupling reactions. Further included are informationally significant peptides made by these methods, wherein the informationally significant peptide is programmed to include non-informational solubility-modifying peptide chains in amino acid sequences of the informationally significant peptides.

Further encompassed by the present invention are kits that may include one or more of the solubility-modified peptides provided herein, and optionally instructions for methods for enhancing protective immunity in a subject that include administering said solubility-modified peptides to said subject, instructions for preparing a solution in accordance with the present invention, and/or one or more additional components of a solution in accordance with the present invention, and/or one or more components used to administer a solution including the solubility-modified peptides to a recipient including for example a syringe.

Although the present disclosure has been described in example embodiments, additional modifications and variations would be apparent to those skilled in the art It is therefore to be understood that the present disclosure herein may be practiced other than as specifically described. Thus, the present embodiments should be considered in all respects as illustrative and not restrictive. Accordingly, it is intended that such changes and modifications fall within the scope of the present disclosure as defined by the claims appended hereto.

We claim:

1. A method comprising
synthesizing solubility-modified sets of one or more peptides comprising
programming insertion of non-informational strings of amino acids of uniform solubility at both C-terminal and N-terminal ends of an informational amimo acid sequence, to allow said informational amino acid sequences to be administered by methods determined by the solubility properties of the non-informational amino acid sequences bonded to their C-terminal and N-terminal ends.

2. A method of making solubility-modified peptides with the requisite solubility properties for tissue delivery by precipitation without extraneous binding and modification of antigenicity, comprising:
incorporating strings of hydrophobic amino acids as solubility modifiers at both C-terminal and N-terminal ends into epitope-containing segments of selected peptides using solid phase peptide synthesis to produced solubility-modified peptides,
wherein said peptides are synthesized to be capable of precipitation within volumes of target tissues of a recipient, when a pharmaceutically acceptable solvent and said solubility modified peptides are administered to a target tissue of said recipient.

3. The method of claim 2, wherein the peptides are overlapping peptides of protein antigens, the method further comprising using solid phase peptide synthesis to incorporate strings of hydrophobic amino acids as solubility modifiers at both the C-terminal and N-terminal ends of into the epitope-containing segments of the overlapping peptides.

4. The method of claim 3, wherein the protein antigens are causes of pathological sensitization.

5. The method of claim 4, wherein the peptides are selected and synthesized specifically for immunotherapy to produce immunomodulation from pathological sensitization to immunological tolerance.

6. The method of claim 2, wherein the peptides are exogenous antigens that are selected and synthesized specifically for treatment or prevention of allergic diseases.

7. The method of claim 2, wherein the peptides are endogenous antigens that are selected and synthesized specifically for treatment or prevention of autoimmune diseases.

8. The method of claim 2, wherein the protein antigens are attributed to causes of pathological tolerance of a cancer or tumor and the protein antigens are selected and synthesized specifically for immunotherapy to produce immunomodulation from pathological tolerance of tumor antigens to protective sensitization.

9. The method of claim 3, wherein the protein antigens are antigens of infectious diseases and the antigens are selected and synthesized specifically for immunotherapy to induce protective sensitization.

10. The method of claim 9, wherein the antigens are selected and synthesized specifically for immunotherapy to induce protective sensitization to diseases to which a recipient has not yet been exposed.

11. The method of claim 9, wherein the antigens are selected and synthesized specifically for immunotherapy to induce protective sensitization to diseases to which a recipient has probably been exposed, but is not known to have been actively infected.

12. The method of claim 9, wherein the antigens are selected and synthesized specifically for immunotherapy to induce protective sensitization to diseases to which a recipient is currently or has previously been infected.

13. The method of claim 12, wherein the disease comprises malaria or TB.

14. The method of claim 9, wherein the disease comprises COVID-19.

15. The method of claim 2, wherein modified peptides are synthesized that sandwich overlapping amino acid sequences from a target antigenic protein between strings of hydrophobic amino acids pre-programmed in place to provide the requisite solubility profile for TDBP, without exposing side chain amino and carboxyl groups to reactions that could result in any form of inactivation.

16. A method of making solubility-modified peptides with the requisite solubility properties for tissue delivery by precipitation without extraneous binding and modification of antigenicity, comprising:
adding solubility-modifying non-epitope sequences of hydrophobic amino acids in a peptide assembly process at or past both ends of epitope-containing MHC-binding sequences, yielding TDBP/VDBP-compatible overlapping peptide sets with fully unblocked epitope amino acid sequences and with necessary solubility properties for VDBP, to produce solubility-modified peptides,
wherein said solubility-modified peptides are synthesized to be capable of precipitation within volumes of target tissues of a recipient, when administered to said target tissue of said patient, as pharmaceutically acceptable solvents.

17. Solubility-modified peptides comprising
insertion of strings of hydrophobic amino acids added by program at both C-terminal and N-terminal ends of peptides to be precipitated into non-liquid tissues by tissue deposition by precipitation.

18. A kit comprising
solubility-modified peptides of claim 17 and
instructions for methods for enhancing protective immunity in a subject that include administering said solubility-modified peptides to said subject.

19. A method of enhancing protective immunity in a subject comprising
administering to a subject a peptide synthesized by the method of claim 2.

20. A method of enhancing protective immunity in a subject comprising
administering to a subject a peptide synthesized by the method of claim 16.

21. A method of making solubility-modified peptides with the requisite solubility properties for tissue delivery by precipitation without extraneous binding and modification of antigenicity, comprising:
using solid phase peptide synthesis to sandwich immunologically active overlapping peptide sequences between solubility-modifying amino acid sequences that were hydrophilic to produce solubility modified peptides,
wherein said peptides are synthesized to be capable of precipitation within volumes of target tissues of a recipient, when a pharmaceutically acceptable solvent and said solubility modified peptides are administered to a target tissue of said recipient.

22. A method comprising
programming non-informational solubility-modifying peptide chains into amino acid sequences of informationally significant peptides to give them solubility properties of being insoluble in water but soluble in a pharmaceutically acceptable water-miscible solvent, without need for post-synthesis or post-hydrolysis coupling reactions.

23. Informationally significant peptides made by the method of claim 22, wherein said informationally significant peptide is programmed to include non-informational solubility-modifying peptide chains in amino acid sequences of said informationally significant peptides.
